(19) United States
(12) Patent Application Publication
(10) Pub. No.: US 2024/0091761 A1
(43) Pub. Date: Mar. 21, 2024

(54) REPLACEABLE FLUID SAMPLING CARTRIDGE FOR CAPTURING A FLUID SAMPLE IN A FLUID SAMPLING SYSTEM

(71) Applicant: Honeywell International Inc., Charlotte, NC (US)

(72) Inventor: Ronald W. MYERS, Dublin, OH (US)

(21) Appl. No.: 17/933,002

(22) Filed: Sep. 16, 2022

(51) Int. Cl. B01L 3/00

(52) U.S. Cl. CPC B01L 3/502; B01L 2300/0838; B01L 2400/0406

(57) ABSTRACT

Various embodiments are directed to a fluid sampling cartridge for capturing a fluid sample and methods of using the same. In various embodiments, a fluid sampling cartridge comprises a fluid conduit having an interior conduit portion, wherein the fluid conduit is configured to receive a volume of fluid at a fluid conduit inlet such that the fluid conduit defines a fluid flow path within the interior conduit portion; a capillary component having a first capillary end in fluid communication with the interior conduit portion and configured to receive at least a portion of the volume of fluid to define a fluid sample; and a cartridge housing defining an interior housing portion, wherein the cartridge housing is configured for engagement with the fluid conduit and the capillary component such that at least a portion of each of the fluid conduit and the capillary component is disposed within the interior housing portion.

### FIELD OF THE INVENTION

Example embodiments of the present disclosure relate generally to analytics of fluid samples and, more particularly, to apparatuses and methods for sampling fluid and analyzing fluid samples.

### BACKGROUND

Applicant has identified many technical challenges and difficulties associated with sampling fluids and analyzing fluid samples. For example, many methods and systems fail to provide an effective mechanism that allows peritoneal dialysis (PD) effluent to be analyzed.

### BRIEF SUMMARY

Various embodiments described herein relate to methods, apparatuses, and systems for sampling fluid and analyzing fluid samples. In accordance with various embodiments of the present disclosure, a fluid sampling cartridge for capturing a fluid sample may comprise a fluid conduit having an interior conduit portion defined between a fluid conduit inlet and a fluid conduit outlet of the fluid conduit, wherein the fluid conduit is configured to receive a volume of fluid at the fluid conduit inlet such that the fluid conduit defines a fluid flow path within the interior conduit portion between the fluid conduit inlet and the fluid conduit outlet; a capillary component having a first capillary end in fluid communication with the interior conduit portion and configured to receive at least a portion of the volume of fluid, the at least a portion of the volume of fluid received by the capillary component from the fluid conduit defining a fluid sample; and a cartridge housing defining an interior housing portion, wherein the cartridge housing is configured for engagement with the fluid conduit and the capillary component such that at least a portion of the fluid conduit and at least a portion of the capillary component are disposed within the interior housing portion.

In various embodiments, the cartridge housing may be configured for selective arrangement in an installed position relative to a fluid sampling system, the installed position being defined by an arrangement of the capillary component relative to an imaging device of the fluid sampling system that enables the imaging device to capture imaging data associated with the fluid sample received within the capillary component. In various embodiments, the cartridge housing may comprise a conduit channel defined within the interior housing portion, the conduit channel being configured to receive at least a portion of the fluid conduit therein to secure the fluid conduit relative to the cartridge housing. In various embodiments, the cartridge housing may comprise a capillary channel defined within the interior housing portion, the conduit channel being configured to receive at least a portion of the capillary component therein to secure the capillary component relative to the cartridge housing. In certain embodiments, the capillary channel may be positioned such that at least a portion of the capillary component provided therein is at least substantially adjacent to a capillary opening defined by the fluid conduit to facilitate the fluid communication between the capillary component and the fluid conduit.

In various embodiments, the cartridge housing may define one or more imaging orifice configured such that at least a portion of the capillary component is visible through the cartridge housing via the one or more imaging orifice. In certain embodiments, the one or more imaging orifice may intersect at least a portion of a capillary channel defined by the cartridge housing, the conduit channel being configured to receive at least a portion of the capillary component therein to secure the capillary component relative to the cartridge housing. In certain embodiments, the one or more imaging orifice may include a first imaging orifice provided on a front surface of the conduit housing and a second imaging orifice provided on a rear surface of the conduit housing. In certain embodiments, the first imaging orifice may be positioned at least substantially adjacent a front capillary surface of the capillary component disposed within the interior housing portion of the cartridge housing such that the front capillary surface is visible through the first imaging orifice, and wherein the second imaging orifice may be positioned at least substantially adjacent a rear capillary surface of the capillary component such that the rear capillary surface is visible through the second imaging orifice.

In various embodiments, the cartridge housing may be defined by a plurality of detachable components configured for selective attachment relative to one another to collectively define the cartridge housing. In certain embodiments, the cartridge housing may comprise a front housing portion and a rear housing portion that are configured to be detachably fastened relative to one another to collectively define an exterior shell of the cartridge housing with the interior housing portion defined therein. In certain embodiments, the front housing portion may define a first conduit channel portion and the rear housing portion defines a second conduit channel portion, the first conduit channel portion and the second conduit channel portion collectively defining a conduit channel within the interior housing portion that is configured for receiving at least a portion of the fluid conduit. In certain embodiments, the front housing portion may define a first capillary channel portion and the rear housing portion defines a second capillary channel portion, the first capillary channel portion and the second capillary channel portion collectively defining a capillary channel within the interior housing portion that is configured for receiving at least a portion of the capillary component. In certain embodiments, the front housing portion may define a first imaging orifice and the rear housing portion defines a second imaging orifice, and wherein at least a portion of the capillary component defining a receiving area within which the capillary component captures the fluid sample is positioned in between the first imaging orifice and the second imaging orifice.

In various embodiments, the fluid conduit may define a capillary opening extending through the conduit sidewall at an intermediate location defined between the fluid conduit inlet and the fluid conduit outlet of the fluid conduit, the capillary opening being configured for receiving at least a portion of the capillary component therethrough. In various embodiments, the capillary component may comprise a first opening defined at a first capillary end and a second opening defined at a second capillary end, wherein the first opening defines a fluid inlet through which the capillary component receives the fluid sample. In certain embodiments, the capillary component may be arranged relative to the fluid conduit such that the first opening of the capillary component is disposed within the interior conduit portion of the fluid conduit.

In various embodiments, the fluid sampling cartridge may be configured for arrangement within fluid sampling system such that the fluid sample captured within the capillary component is disposed within a field of view of an imaging device. In certain embodiments, the fluid sampling cartridge may be configured to enable an imaging of the fluid sample captured within the capillary component by the imaging device at an instance during operation of the fluid sampling system in which the volume of fluid is flowing through the fluid conduit. In various embodiments, the capillary component may define an at least substantially perpendicular arrangement relative to the fluid conduit.

The foregoing illustrative summary, as well as other exemplary objectives and/or advantages of the disclosure, and the manner in which the same are accomplished, are further explained in the following detailed description and its accompanying drawings.

### BRIEF DESCRIPTION OF THE DRAWINGS

The description of the illustrative embodiments may be read in conjunction with the accompanying figures. It will be appreciated that, for simplicity and clarity of illustration, elements illustrated in the figures have not necessarily been drawn to scale, unless described otherwise. For example, the dimensions of some of the elements may be exaggerated relative to other elements, unless described otherwise. Embodiments incorporating teachings of the present disclosure are shown and described with respect to the figures presented herein, in which:

FIG. 1 illustrates an example fluid sampling and analyzing system in accordance with some example embodiments described herein;

FIG. 2 illustrates a cross-sectional view of an exemplary fluid sampling system in accordance with various embodiments described herein;

FIG. 3 illustrates a schematic representation of an example fluid sampling system in accordance with some example embodiments described herein;

FIG. 4 illustrates a perspective view of an example replaceable fluid sampling cartridge in accordance with some example embodiments described herein;

FIG. 5 illustrates an exploded view of an example replaceable fluid sampling cartridge in accordance with some example embodiments described herein;

FIGS. 6A and 6B illustrate perspective views of a replaceable fluid sampling cartridge in accordance with various embodiments described herein; and

FIG. 7 illustrates an isolated perspective view of various components of a fluid sampling system and an example replaceable fluid sampling cartridge in accordance with some example embodiments described herein.

### DETAILED DESCRIPTION OF THE INVENTION

Some embodiments of the present disclosure will now be described more fully hereinafter with reference to the accompanying drawings, in which some, but not all embodiments of the disclosure are shown. Indeed, these disclosures may be embodied in many different forms and should not be construed as limited to the embodiments set forth herein; rather, these embodiments are provided so that this disclosure will satisfy applicable legal requirements. Like numbers refer to like elements throughout.

As used herein, terms such as “front,” “rear,” “top,” etc. are used for explanatory purposes in the examples provided below to describe the relative position of certain components or portions of components. Furthermore, as would be evident to one of ordinary skill in the art in light of the present disclosure, the terms “substantially” and “approximately” indicate that the referenced element or associated description is accurate to within applicable engineering tolerances.

As used herein, the term “comprising” means including but not limited to and should be interpreted in the manner it is typically used in the patent context. Use of broader terms such as comprises, includes, and having should be understood to provide support for narrower terms such as consisting of, consisting essentially of, and comprised substantially of.

The phrases “in one embodiment,” “according to one embodiment,” and the like generally mean that the particular feature, structure, or characteristic following the phrase may be included in at least one embodiment of the present disclosure, and may be included in more than one embodiment of the present disclosure (importantly, such phrases do not necessarily refer to the same embodiment).

The word “example” or “exemplary” is used herein to mean “serving as an example, instance, or illustration.” Any implementation described herein as “exemplary” is not necessarily to be construed as preferred or advantageous over other implementations.

If the specification states a component or feature “may,” “can,” “could,” “should,” “would,” “preferably,” “possibly,” “typically,” “optionally,” “for example,” “often,” or “might” (or other such language) be included or have a characteristic, that a specific component or feature is not required to be included or to have the characteristic. Such a component or feature may be optionally included in some embodiments, or it may be excluded.

The term “electronically coupled,” “electronically coupling,” “electronically couple,” “in communication with,” “in electronic communication with,” or “connected” in the present disclosure refers to two or more elements or components being connected through wired means and/or wireless means, such that signals, electrical voltage/current, data and/or information may be transmitted to and/or received from these elements or components.

As described above, there are many technical challenges and difficulties associated with sampling fluids and analyzing fluid samples. For example, there are many technical challenges and difficulties related to medical analysis in renal care.

“Renal care” refers to medical care that provides diagnosis and treatment associated with the kidney (including but not limited to, chronic renal disease and/or acute renal disease). For example, when the patient's kidney stops working properly, renal care may include, but not limited to, performing dialysis procedures. The dialysis procedures are designed to remove waste products and excess fluids from the blood of the patient, and therefore improve the health of the patient.

Renal care is a huge federal expenditure, and medical care reimbursement guidelines are changing to shift more patients away from traditional hemodialysis (HD) to peritoneal dialysis (PD). In particular, PD provides a mechanism to remove waste products from a patient's blood when the patient's kidneys cannot adequately function, and PD is different from HD.

In particular, during an example PD procedure, a cleansing fluid (such as, but not limited to, a dialysis solution such as water with sugar and other additive) flows through a tube or a pipe (such as, but not limited to, a catheter) into the patient's body. More specifically, the cleansing fluid is injected into a part of a patient's abdomen. When the cleansing fluid is inside the patient's body, the cleansing fluid absorbs waste products from the patient's body. The lining of the abdomen (also known as peritoneum) can act as a filter and remove waste products from the patient's blood. After a set period of time, the fluid with the filtered waste products (referred herein as peritoneal dialysis (PD) effluent) flows out of the patient's abdomen and can be discarded.

In many instances, PD can be done by a patient at home. For example, a patient may use a PD machine to conduct PD procedure while the patient is sleeping. In contrast, HD must be performed in a health clinic by a trained healthcare professional. As such, PD can cost substantially less than HD, and can provide cost saving benefits.

However, PD procedures are faced with some drawbacks. One of the drawbacks is that patients who undergo PD may develop infections, which can force patients to switch back to HD. As such, early detection of infections after a patient undergoes PD can be beneficial for alerting patients, as well as care providers, so that early action can be taken to limit the severity and frequency of infections.

Various embodiments of the present disclosures overcome technical challenges and difficulties associated with sampling fluids and analyzing fluid samples, and address drawbacks faced by PD procedures. For example, various embodiments of the present disclosures include a fluid sampling and analyzing system in the field of peritoneal dialysis that provides an effective mechanism to sample and analyze PD effluent. For example, various embodiments of the present disclosure provide a fluid sampling system that can take sampling images of the PD effluent. The sampling images of the PD effluent are visually assessed to determine cell counts (such as, but not limited to, white blood cell count) of the fluid. For example, the more white blood cells that there are in the PD effluent, the more likely that the patient is having an infection as the white blood cells make the PD effluent cloudy. As such, various embodiments of the present disclosure can detect indicators of infections based on the PD effluent, and can have the potential to detect infections earlier (which can lead to better patient outcomes) and provide better specificity in the detection results (for example, based on the concentrations of white blood cells and/or types of white blood cells).

In some embodiments, the fluid sampling system works in tandem with a PD machine (also referred to as a “cycler”). For example, the fluid sampling system may be integrated into the PD machine. Additionally, or alternatively, the fluid sampling system may operate as a stand-alone device that is connected to the fluid conduit from the PD machine to receive the PD effluent. In particular, the fluid sampling system comprises a fluid imaging chamber that is disposable and/or removable from the fluid sampling system. As the PD effluent is pumped out of the patient's body, some of the fluid passes through the fluid imaging chamber. The fluid sampling system may also include an image sensor component that can generate digital holography image data of the PD effluent.

Various embodiments of the present disclosure relate to replaceable fluid sampling cartridges that may be configured for use in a fluid sampling system (e.g., a “PD machine”) to capture a fluid sample from a volume of fluid as it flows through the fluid sampling system. The replaceable fluid sampling cartridge may be configured to receive the volume of fluid and capture the fluid sample therefrom as the volume of fluid is flowing through the cartridge, such that the collection of the fluid sample does not interrupt the operation of the fluid sampling system. Further, in various embodiments, the replaceable fluid sampling cartridge described herein includes at least one imaging orifices defined within the cartridge housing that provide a direct line of sight to the fluid sample captured by the fluid sampling cartridge through the cartridge housing. As described herein, such a configuration allows an imaging device of the fluid sampling system to capture images of the captured fluid sample without requiring the fluid sampling cartridge to be disassembled and/or removed from the fluid sampling system during operation.

Further, the present invention allows for the fluid sampling system to execute a fluid sample analysis operation associated with the fluid sample captured by the replaceable fluid sampling cartridge without interrupting the flow of the volume of fluid through the fluid sampling cartridge. For example, in some embodiments, after the digital holography image data is generated based on the image of the fluid sample captured within the replaceable fluid sampling cartridge, the fluid sampling system may upload the digital holography image data to a remote computing platform. In some embodiments, the digital holography image data comprises digital holography image(s) of the PD effluent, and the remote computing platform can computationally generate reconstructed/focused image(s) based on the digital holography image(s). In some embodiments, estimated sample characteristics data associated with the fluid sample can be determined based on the reconstructed/focused image(s). For example, the reconstructed/focused image(s) can be provided to one or more machine learning models to detect, count, classify, and/or measure the sizes of the detected particles and cells from the PD effluent as shown in the reconstructed/focused image(s). In some embodiments, the results (e.g. estimated sample characteristics data) can be provided to the end user (for example, patients, healthcare providers, etc.), enabling near-real-time analysis of the PD fluid contents and detection of infection.

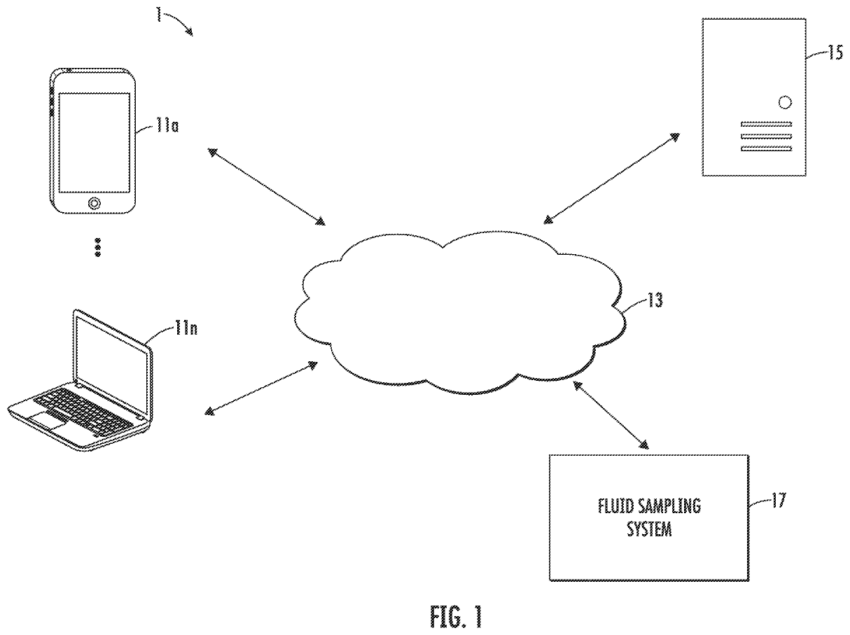

Referring now to FIG. 1, an example diagram illustrating an example fluid sampling and analyzing system in accordance with some example embodiments described herein is provided.

As shown in FIG. 1, the example fluid sampling and analyzing system 1 may comprise apparatuses, devices, and components such as, but not limited to, a fluid sampling system 17, one or more mobile computing devices 11A . . . 11N, a remote computing server 15 in a remote computing platform, and one or more networks 13. In various embodiments, as described herein, an exemplary fluid sampling system 17 of an exemplary fluid sampling and analyzing system 1 may be configured to execute both a fluid sample collection operation and a fluid sample analysis operation to determine one or more fluid characteristics associated with a volume of fluid. For example, a fluid sample may be captured from the volume of fluid as part of the fluid sample collection operation, and the fluid sampling system 17 may further visually analyze the fluid sample using imaging data associated with the fluid sample (e.g., an image of the fluid sample), as described herein. In various embodiments, an exemplary replaceable fluid sampling cartridge may be utilized during operation of the fluid sampling system 17 to capture the fluid sample from a volume of fluid and facilitate the imaging of the fluid sample by an imaging device of the fluid sampling system 17.

While the description above provides an example fluid sampling and analyzing system, it is noted that the scope of the present disclosure is not limited to the description above. In some examples, an example fluid sampling and analyzing system 1 may comprise one or more additional and/or alternative elements. For example, an example fluid sampling and analyzing system in accordance with embodiments of the present disclosure may comprise more than one fluid sampling system. Additionally, or alternatively, an example fluid sampling and analyzing system in accordance with embodiments of the present disclosure may comprise more than one remote computing server and/or more than one remote computing platform. Additionally, or alternatively, an example fluid sampling and analyzing system in accordance with embodiments of the present disclosure may comprise a replaceable fluid sampling cartridge.

In some embodiments, each of the components of the example fluid sampling and analyzing system 1 may be in electronic communication with, for example, one another over the same or different wireless or wired networks 13 including, for example, a wired or wireless Personal Area Network (PAN), Local Area Network (LAN), Metropolitan Area Network (MAN), Wide Area Network (WAN), and/or the like. Additionally, while FIG. 1 illustrates certain system entities as separate, standalone entities, the various embodiments are not limited to this particular architecture.

For example, the fluid sampling system 17, one or more mobile computing devices 11A . . . 11N, the remote computing server 15 in the remote computing platform may be in electronic communication with one another to exchange data and information. As defined herein, the fluid sampling system 17 may receive a fluid sample (such as, but not limited to, peritoneal dialysis effluent, urine, oil, and/or the like) and may generate digital holography image data associated with the fluid sample. In some embodiments, the fluid sampling system 17 may transmit the digital holography image data to the one or more mobile computing devices 11A . . . 11N and/or the remote computing server 15 in the remote computing platform, and the one or more mobile computing devices 11A. 11N and/or the remote computing server 15 in the remote computing platform may determine estimated sample characteristics data associated with the fluid sample based at least in part on the digital holography image data. In some embodiments, the estimated sample characteristics data comprises an estimated concentration level of white blood cells within the fluid sample, estimated size values of particles within the fluid sample, and/or the like. In some embodiments, the one or more mobile computing devices 11A . . . 11N and/or the remote computing server 15 may transmit the estimated sample characteristics data to another device (such as, but not limited to, the fluid sampling system 17, one of the one or more mobile computing devices 11A . . . 11N, and/or another remote computing server in the remote computing platform)

Additionally, or alternatively, the fluid sampling system 17 may determine estimated sample characteristics data associated with the fluid sample based at least in part on the digital holography image data. In some embodiments, the estimated sample characteristics data comprises an estimated concentration level of white blood cells within the fluid sample, estimated size values of particles within the fluid sample, and/or the like. In some embodiments, the fluid sampling system 17 may transmit the estimated sample characteristics data to another device (such as, but not limited to, the one or more mobile computing devices 11A . . . 11N and/or the remote computing server 15).

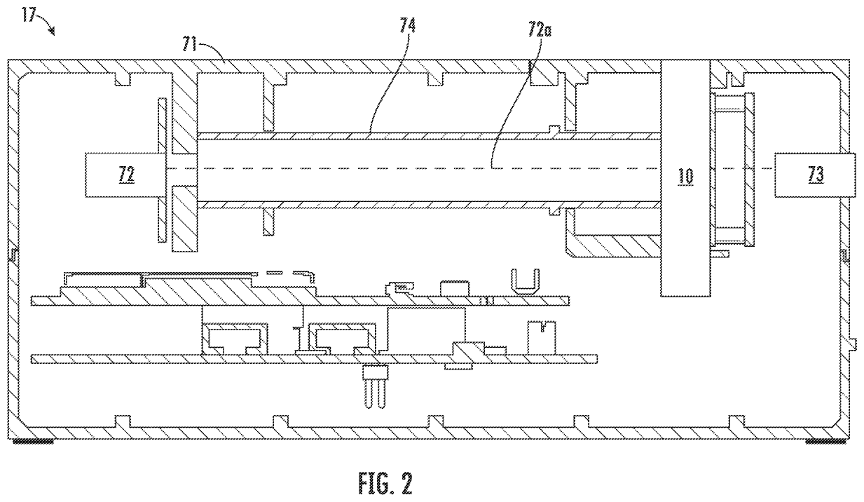

Referring now to FIG. 2, a schematic representation of an example fluid sampling system in accordance with some example embodiments described herein. In particular, FIG. 2 illustrates a schematic representation of a cross-section of an example fluid sampling system 17. In various embodiments, the example fluid sampling system 17 may embody a machine configured to execute a fluid sample collection operation and a fluid sample analysis operation in order to determine one or more fluid characteristics associated with a volume of fluid. As illustrated, the fluid sampling system 17 may be configured to receive a replaceable fluid sampling cartridge 10 and facilitate an interaction between the replaceable fluid sampling cartridge 10 and a volume of fluid received by the fluid sampling system 17 that allows the replaceable fluid sampling cartridge 10 to capture a fluid sample from the volume of fluid, as described herein. For example, the replaceable fluid sampling cartridge 10 may be provided in an installed position within the fluid sampling system in-line between a fluid inlet and a fluid outlet of the fluid sampling system 17. For example, in such an exemplary configuration, a cartridge fluid inlet defined by the replaceable fluid sampling cartridge 10 may be fluidly connected to the fluid inlet of the fluid sampling system 17 and a cartridge fluid outlet defined by the replaceable fluid sampling cartridge 10 is fluidly connected to the fluid outlet of the fluid sampling system 17. Further, the fluid sampling system 17 may be configured such that, in the installed position, the replaceable fluid sampling cartridge 10 is disposed in between an illumination device 72 and an imaging device 73. As illustrated, the fluid sampling system 17 may be configured such that, upon the replaceable fluid sampling cartridge 10 having captured a fluid sample (e.g., within a capillary component) from the volume of fluid flowing through the fluid sampling system 17, a fluid sample analysis operation may be executed by the fluid sampling system 17, such as, for example, by using the illumination device 72 to illuminate at least a portion of the fluid sample within the replaceable fluid sampling cartridge 10 and capturing an image of the illuminated fluid sample with the imaging device 73 while the replaceable fluid sampling cartridge 10 is still disposed in the installed position in the fluid sampling system 17.

For example, in various embodiments, an example fluid sampling system 17 may be configured to receive different volumes of fluid from which one or more replaceable fluid sampling cartridges 10 may capture a respective fluid sample. In some embodiments, the fluid sample is associated with PD effluent. For example, the fluid sample may comprise PD effluent. In the present disclosure, the term “PD effluent” refers to a liquid that is discharged from a PD procedure. For example, the PD effluent may be a liquid that is discharged from a patient's body as an end product from performing a PD procedure on the patient. As described above, a dialysis solution is injected into the patient's body when a PD procedure is performed on the patient. The dialysis solution dwells within the patient's body and eventually is discharged as a PD effluent. In some embodiments, the PD effluent may be received from a PD machine. For example, a discharging conduit of the PD machine that discharges fluids from the PD procedure can be connected to the fluidic inlet 210. In such an example, the fluid discharged from the PD machine is the PD effluent. As described above, the fluidic outlet 212 may be connected to a fluid output conduit to discharge the PD effluent.

While the description above provides an example of a fluid sample, it is noted that the scope of the present disclosure is not limited to the description above. In some examples, an example fluid sample may comprise one or more additional and/or alternative fluids. For example, the fluid sample may comprise urine. Additionally, or alternatively, the fluid sample may comprise oil. Additionally, or alternatively, the fluid sample may comprise blood.

As described herein, in various embodiments, the fluid composition sensor 10 may comprise an illumination device 72 configured to emit one or more light beams in order to facilitate a more effective and/or efficient imaging of the fluid sample captured by the replaceable fluid sampling cartridge 10. For example, in various embodiments, the illumination device 72 may be a laser, lamp, light-emitting diode (LED), and/or the like, which may be collectively configured to generate a light beam (e.g., ultraviolet, visible, infrared, white, a single visible color, or multiple color light) that may be emitted toward the replaceable fluid sampling cartridge 10, as described herein in further detail. An illumination device 72 of the fluid sampling system 17 may be configured to emit one or more light beams so as to engage the capillary component defined by the replaceable fluid sampling cartridge 10 and illuminate at least a portion of the fluid sample captured therein. In various embodiments, as illustrated in FIG. 2, the fluid sampling system 17 may be configured such that the illumination device 72 is at least substantially aligned with the imaging device 73. Further, for example, the illumination device 72 may be arranged such that the light beam emitted therefrom extends through an imaging orifice defined by the replaceable fluid sampling cartridge 10 in a direction that is at least substantially aligned with capillary component housing within a cartridge housing of the replaceable fluid sampling cartridge 10, such that at least a portion of the one or more light beams illuminate the fluid sample disposed within the capillary component. As described herein, an imaging device 73 defined within the fluid sampling system 17 may be configured to utilize the light beam emitted from the illumination device 72 in order to capture an image of the fluid sample received by the capillary component of the replaceable fluid sampling cartridge 10 using one or more imaging techniques such as, for example holographic microscopy (e.g., lensless holography) and/or the like.

In some embodiments, the example fluid sampling system 17 may comprise a shade covering 74. In some embodiments, the shade covering 74 comprises materials that block light (such as, but not limited to, opaque materials). For example, the shade covering 74 may prevent stray light from interfering with the at least one light beam emitted by the illumination device 72.

While the description above provides an example of positional relationships between the illumination device 72, the imaging device 73, and an exemplary replaceable fluid sampling cartridge 10 provided in the installed position, it is noted that the scope of the present disclosure is not limited to the description above. In some examples, an example illumination device 72 and/or an example imaging device 73 may be positioned differently with respect to the installed position defined by the exemplary replaceable fluid sampling cartridge 10 shown in FIG. 2.

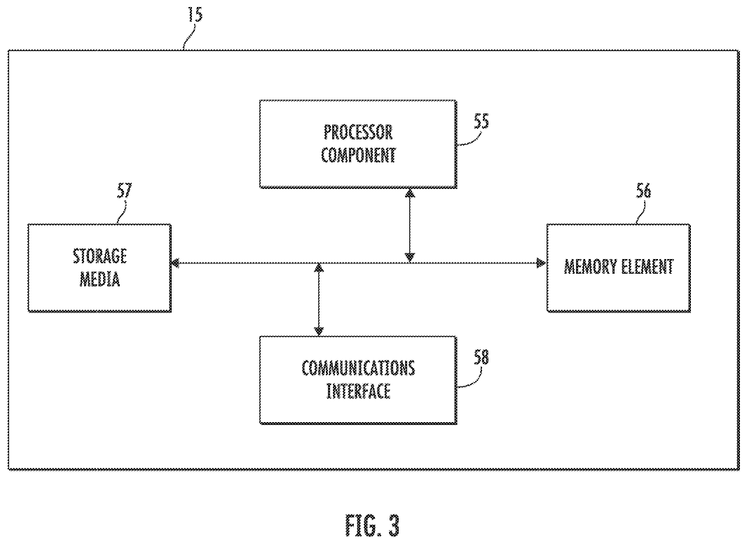

Referring now to FIG. 3, an example schematic representation of an example remote computing server in an example remote computing platform in accordance with some example embodiments described herein. In some embodiments, the example remote computing platform may be a cloud computing platform, and the example remote computing server may be a cloud computing server.

As indicated, in some embodiments, the remote computing server 15 may include one or more network and/or communications interface 48 for communicating with various computing entities, such as by communicating data, content, information, and/or similar terms used herein that can be transmitted, received, operated on, processed, displayed, stored, and/or the like. For instance, the remote computing server 15 may communicate with the fluid sampling system 17, one or more mobile computing devices 11A . . . 11N, and/or the like.

As shown in FIG. 3, in one embodiment, the remote computing server 15 may include or be in communication with one or more processor components (for example, processor component 55) (also referred to as processors, processing circuitry, and/or similar terms used herein interchangeably) that communicate with other elements within the remote computing server 15 via a bus, for example, or network connection. As will be understood, the processor component 55 may be embodied in a number of different ways. For example, the processor component 55 may be embodied as one or more complex programmable logic devices (CPLDs), microprocessors, multi-core processors, co-processing entities, application-specific instruction-set processors (ASIPs), and/or controllers. Further, the processor component 405 may be embodied as one or more other processing devices or circuitry. The term circuitry may refer to an entirely hardware embodiment or a combination of hardware and computer program products. Thus, the processor component 55 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processor component 55 may be configured for a particular use or configured to execute instructions stored in volatile or non-volatile media or otherwise accessible to the processor component 55. As such, whether configured by hardware or computer program products, or by a combination thereof, the processor component 55 may be capable of performing steps or operations according to embodiments of the present disclosure when configured accordingly.

In one embodiment, the remote computing server 15 may further include or be in communication with volatile media (also referred to as volatile storage, memory, memory storage, memory circuitry and/or similar terms used herein interchangeably). In one embodiment, the volatile storage or memory may also include one or more memory element 56 as described above, such as RAM, DRAM, SRAM, FPM DRAM, EDO DRAM, SDRAM, DDR SDRAM, DDR2 SDRAM, DDR3 SDRAM, RDRAM, RIMM, DEVIM, SIMM, VRAM, cache memory, register memory, and/or the like. As will be recognized, the volatile storage or memory element 56 may be used to store at least portions of the databases, database instances, database management system entities, data, applications, programs, program modules, scripts, source code, object code, byte code, compiled code, interpreted code, machine code, executable instructions, and/or the like being executed by, for example, the processor component 55 as shown in FIG. 3. Thus, the databases, database instances, database management system entities, data, applications, programs, program modules, scripts, source code, object code, byte code, compiled code, interpreted code, machine code, executable instructions, and/or the like may be used to control certain aspects of the operation of the remote computing server 15 with the assistance of the processor component 55 and operating system.

In one embodiment, the remote computing server 15 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or storage media 57 as described above, such as hard disks, ROM, PROM, EPROM, EEPROM, flash memory, MMCs, SD memory cards, Memory Sticks, CBRAM, PRAM, FeRAM, RRAM, SONOS, racetrack memory, and/or the like. As will be recognized, the non-volatile storage or storage media 57 may store databases, database instances, database management system entities, data, applications, programs, program modules, scripts, source code, object code, byte code, compiled code, interpreted code, machine code, executable instructions, and/or the like. The term database, database instance, database management system entity, and/or similar terms used herein interchangeably and in a general sense to refer to a structured or unstructured collection of information/data that is stored in a computer-readable storage medium.

Storage media 57 may also be embodied as a data storage device or devices, as a separate database server or servers, or as a combination of data storage devices and separate database servers. Further, in some embodiments, storage media 57 may be embodied as a distributed repository such that some of the stored information/data is stored centrally in a location within the system and other information/data is stored in one or more remote locations. Alternatively, in some embodiments, the distributed repository may be distributed over a plurality of remote storage locations only. An example of the embodiments contemplated herein would include a cloud data storage system maintained by a third-party provider and where some or all of the information/data required for the operation of the recovery prediction system may be stored.

As indicated, in one embodiment, the remote computing server 55 may also include one or more network and/or communications interface 58 for communicating with various computing entities, such as by communicating data, content, information, and/or similar terms used herein interchangeably that can be transmitted, received, operated on, processed, displayed, stored, and/or the like.

As indicated, in one embodiment, the remote computing server 55 may also include one or more network and/or communications interface 58 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOC SIS), or any other wired transmission protocol. Similarly, the remote computing server 55 may be configured to communicate via wireless external communication networks using any of a variety of protocols, such as general packet radio service (GPRS), Universal Mobile Telecommunications System (UMTS), Code Division Multiple Access 1900 (CDMA1900), CDMA1900 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), Institute of Electrical and Electronics Engineers (IEEE) 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol. The remote computing server 105 may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

As will be appreciated, one or more of the remote computing server's components may be located remotely from components of other remote computing servers, such as in a distributed system. Furthermore, one or more of the components may be aggregated and additional components performing functions described herein may be included in the remote computing server 155. Thus, the remote computing server 105 can be adapted to accommodate a variety of needs and circumstances.

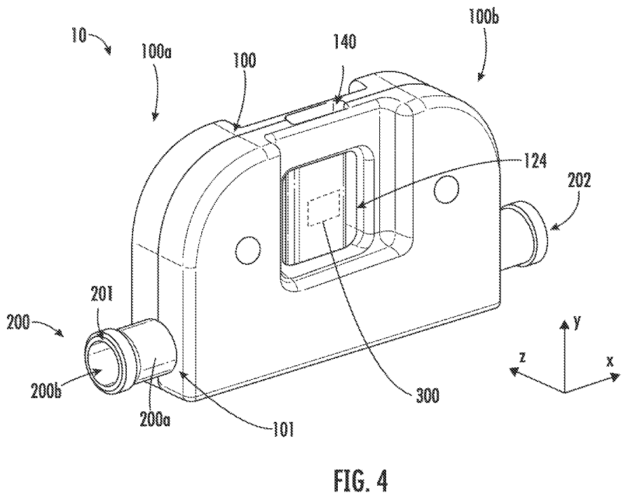

Referring now to FIG. 4, a perspective view of an example replaceable fluid sampling cartridge 10 in accordance with some example embodiments described herein is provided. In various embodiments, a replaceable fluid sampling cartridge may be configured for receiving a fluid sample from a volume of fluid within a fluid sampling system. As illustrated in FIG. 4, an exemplary replaceable fluid sampling cartridge 10 may comprise a cartridge housing 100, a fluid conduit 200, and a capillary component 300. In various embodiments, the fluid conduit 200 may embody a tubular component configured for receiving a volume of fluid flowing through a fluid sampling system. For example, the fluid conduit 200 may have an interior conduit portion 200b defined within a conduit sidewall 200a. The interior conduit portion 200b may be defined between a fluid conduit inlet 201 and a fluid conduit outlet 202 of the fluid conduit 200, each of which may be configured for selective engagement with a respective adjacent conduit portion of a fluid sampling system (e.g., fluid sampling system 17 shown in FIG. 2) to establish a fluid connection therewith and facilitate the arrangement of the replaceable fluid sampling cartridge 10 in an installed position relative to the fluid sampling system. As described herein, the fluid conduit 200 may define a fluid flow path within the interior conduit portion 200b between the fluid conduit inlet 201 and the fluid conduit outlet 202 along which at least a portion of the volume of fluid received by the fluid conduit 200 travels through the replaceable fluid sampling cartridge 10. Further, in various embodiments, as described herein, the fluid conduit 200 may be configured to facilitate the delivery of at least a portion of the volume of fluid flowing through the interior conduit portion 200b to an exemplary capillary component 300 fluidly and/or mechanically connected thereto.

In various embodiments, a capillary component 300 of an exemplary replaceable fluid sampling cartridge 10 may be in fluid communication with the interior conduit portion 200b of the fluid conduit 200 such that the capillary component 300 is configured to capture a fluid sample for imaging and/or analysis by an exemplary fluid sampling system by receiving at least a portion of the volume of fluid within the interior conduit portion 200b. For example, the capillary component 300 may have a first capillary end that is in fluid communication with the interior conduit portion 200b such that as the volume of fluid received by the fluid conduit 200 (e.g., the fluid conduit inlet 201) flows along the fluid flow path defined by the interior conduit portion 200b, at least a portion of the volume of fluid engaged with and/or provided at least substantially adjacent to the first capillary end of the capillary component 300 may be diverted into, absorbed by, and/or otherwise received by the capillary component 300 via a capillary effect and/or a capillary action (e.g., via an at least substantially small opening defined at the first capillary end) so as to define a fluid sample captured by and/or disposed at the capillary component 300. The terms “capillary effect” and “capillary action” refer to the ability of a capillary component 300 to draw fluid to flow or expand through part(s) or all of the capillary component 300 without the assistance of (or, in some example, even in opposition to) an external force. As described in further detail herein, an exemplary capillary component 300 may be defined at least in part by a capillary component length defined between the first capillary end and an opposing second capillary end, and may be configured such that at least a portion of the fluid sample captured thereby remains disposed in a position (e.g., in a receiving area) defined between the first and second capillary ends to facilitate the imaging of the captured fluid sample by the fluid sampling system.

In various embodiments, a cartridge housing 100 of an exemplary replaceable fluid sampling cartridge 10 may embody an exterior shell of the replaceable fluid sampling cartridge 10 that defines an interior housing portion therein. For example, the cartridge housing 100 may define at least a portion of the exterior of the replaceable fluid sampling cartridge 10. An exemplary cartridge housing 100 may be defined at least in part by a cartridge height (e.g., measured in the y-direction as defined in the exemplary orientation illustrated in FIG. 4) and a cartridge width (e.g., measured in the x-direction as defined in the exemplary orientation illustrated in FIG. 4), the cartridge width being defined by the distance between opposing lateral ends 100a, 100b of the cartridge housing 100. length, and a width, wherein the length of the housing 100 is defined by the distance between a first end and a second end.

As illustrated in FIG. 4, the cartridge housing 100 may be configured such that at least a portion of one or more components of the replaceable fluid sampling cartridge 10, such as, for example, the fluid conduit 200 and/or the capillary component 300, may be housed, secured, and/or otherwise disposed within the interior housing portion in order to facilitate operation of the replaceable fluid sampling cartridge 10. For example, as illustrated in FIG. 4, in various embodiments, the cartridge housing 100 may be configured for engagement with the fluid conduit 200 and the capillary component 300 such that at least a portion of the fluid conduit 200 and at least a portion of the capillary component 300 are disposed within the interior housing portion defined by the cartridge housing 100. Further, as described herein, the cartridge housing 100 may be configured for selective arrangement in an installed position relative to an exemplary fluid sampling system to facilitate the capture of a fluid sample within the replaceable fluid sampling cartridge 10 during operation of the fluid sampling system. The cartridge housing 100 may be configured to facilitate an engagement with one or more components of the fluid sampling system to at least partially secure the replaceable fluid sampling cartridge 10 in the installed position relative to the fluid sampling system. In various embodiments, a replaceable fluid sampling cartridge 10 being in the installed position may be defined by an arrangement of the cartridge housing 100 that causes the capillary component 300 secured therein to be positioned relative to an imaging device of the fluid sampling system such that the imaging device may capture imaging data associated with a fluid sample received within the capillary component 300. In various embodiments, the cartridge housing 100 of an exemplary replaceable fluid sampling cartridge 10 may be made of an at least substantially rigid material, such as, for example, a plastic material, a metal material, a composite material, and/or the like, or any other material suitable for securing the positions of the fluid conduit 200 and the capillary component 300 and facilitating the arrangement of the replaceable fluid sampling cartridge 10 in an installed position, as described herein.

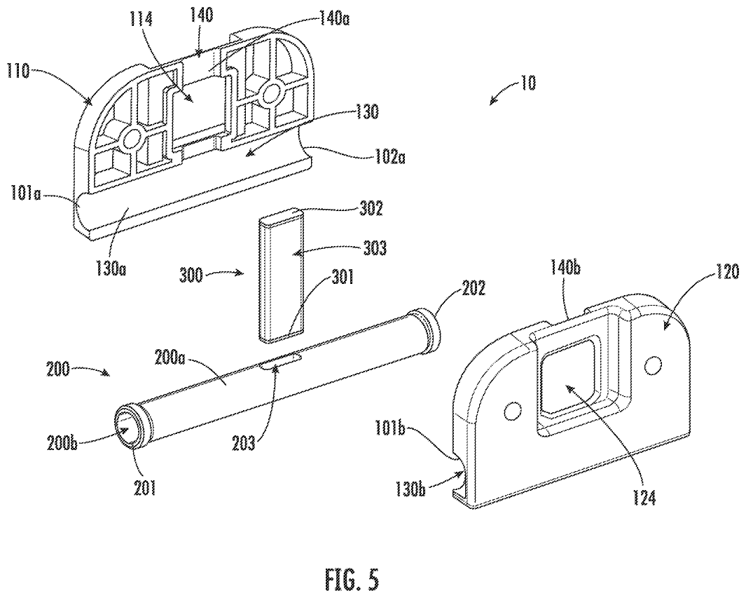

In various embodiments, an exemplary capillary component 300 may be configured to capture a fluid sample from a static volume of fluid defined within the fluid sampling system. Although FIGS. 4 and 5 illustrate exemplary embodiments wherein the capillary component 300 of the replaceable fluid sampling cartridge 10 is configured to capture a fluid sample from a volume of fluid flowing through a fluid conduit 200 (e.g., between the conduit fluid inlet 201 and the conduit fluid outlet 202), it should be understood that, in various embodiments, an exemplary replaceable fluid sampling cartridge 10 may be configured to enable the capture of a fluid sample using a capillary effect or capillary action to pull the fluid sample from the static volume of fluid into the capillary component 300 (e.g., the viewing area defined therein). For example, in various embodiments, an exemplary replaceable fluid sampling cartridge 10 configured to capture a fluid sample from a static volume of fluid may include a capillary component 300 that protrudes out of the cartridge housing 100 such that having a first capillary end of the capillary component 300 may be selectively engaged (e.g., dipped into) with the static volume of fluid. For example, in such an exemplary circumstance, the static volume of fluid may be defined within a container positioned within the fluid sampling system such that a replaceable fluid sampling cartridge 10 positioned in the installed position may pull a fluid sample from the fluid container into the capillary component 300 (e.g., via a capillary effect and/or capillary action).

Referring now to FIG. 5, an exploded view of an example replaceable fluid sampling cartridge 10 in accordance with some example embodiments described herein is provided. As shown, the cartridge housing 100 of an exemplary replaceable fluid sampling cartridge 10 may comprise a conduit channel 130 defined within the interior housing portion that is configured to receive at least a portion of a fluid conduit 200 therein to secure the fluid conduit 200 relative to the cartridge housing 100. For example, the conduit channel 130 of the cartridge housing 100 may be defined by one or more surfaces (e.g., curved surfaces) and/or material recesses having a configuration corresponding to that of the conduit sidewall 200a (e.g., a at least substantially cylindrical configuration) such that the fluid conduit 200 may be positioned within the conduit channel 130. In various embodiments, the conduit channel 130 may define a channel length that extends along the cartridge width of the cartridge housing 100 between the first and second lateral ends 100a, 100b of the cartridge housing 100. As such, the cartridge housing 100 may be configured for engagement with the fluid conduit 200 at the conduit channel 130 such that at least a portion of the conduit length of the fluid conduit 200 is positioned within the interior housing portion between the first and second lateral ends 100a, 100b of the housing 100 (e.g., between the first conduit opening 101 and a second conduit opening 102).

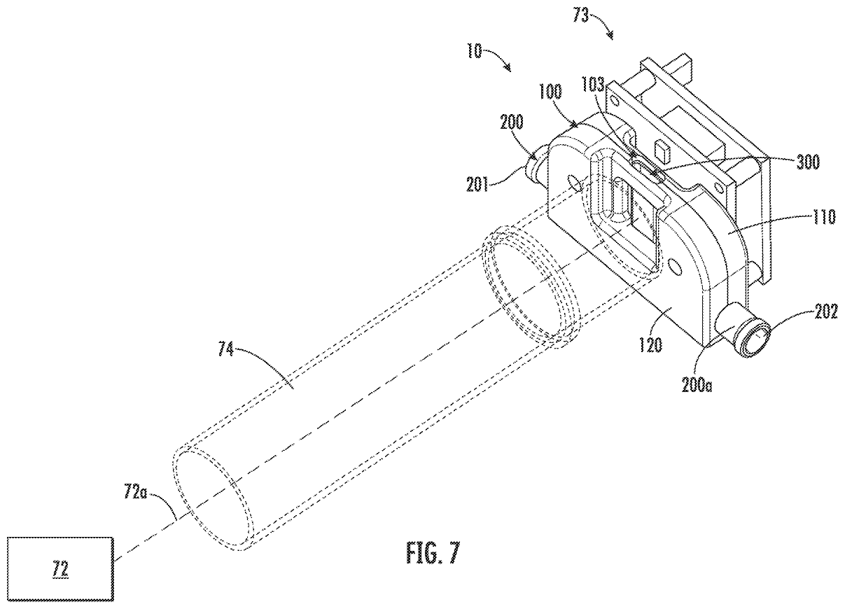

In various embodiments, the cartridge housing 100 of the exemplary replaceable fluid sampling cartridge 10 may further comprise a capillary channel 140 defined within the interior housing portion that is configured to receive at least a portion of a capillary component 300 therein to secure the capillary component 300 relative to the cartridge housing 100. For example, the capillary channel 140 of the cartridge housing 100 may be defined by one or more surfaces and/or material recesses having a configuration corresponding to that of the capillary component 300 such that the capillary component 300 may be positioned within the capillary channel 140. In various embodiments, the capillary channel 140 may define a channel length that extends in a direction at least substantially perpendicular to the channel length of the conduit channel 130, such as, for example, along the cartridge height of the cartridge housing 100. For example, as described in further detail herein, the cartridge housing 100 may be configured for engagement with the capillary component 300 at the capillary channel 140 such that at least a portion of the capillary length of the capillary component 300 may be positioned within the interior housing portion at least substantially adjacent the fluid conduit 200 to facilitate a fluid connection between the capillary component 300 and the fluid conduit 200 (e.g., via a capillary opening 203 defined in the conduit sidewall 200a). For example, the capillary channel 140 may be arranged in a perpendicular configuration relative to the conduit channel 130 such that the capillary channel 140 extends from a first channel end defined at an intermediate location along the length of the conduit channel 130 (e.g., between the first and second channel openings 101, 102) perpendicularly away from the conduit channel to a second conduit end defined at a top end of the cartridge housing 100. In various embodiments, the second conduit end of the capillary channel 140 defined at the top end of the cartridge housing 100 may embody a third channel opening (e.g., third channel opening 103 as shown in the exemplary embodiment of FIG. 7) that facilitates a fluid connection (e.g., an airflow) between an external environment (e.g., outside of the cartridge housing 100) and a capillary component 300 positioned in the interior housing portion within the capillary channel 140.

In various embodiments, the capillary channel 140 may be at least partially aligned and/or intersected with an imaging orifice 114 defined by the cartridge housing 100 such that, upon the capillary component 300 being positioned within the capillary channel 140, at least a portion of the capillary component 300 is visible from outside the cartridge housing 100 via the imaging orifice 114. For example, in such an exemplary circumstance, the capillary cannel 140 may be defined by a plurality of discontinuous channel portions arranged in an aligned configuration (e.g., centered along a common axis) separated by the opening of the imaging orifice 114 defined by the cartridge housing 100.

In various embodiments, the cartridge housing 100 of the exemplary replaceable fluid sampling cartridge 10 may further comprise at least one imaging orifice extending therethrough so as to facilitate and/or enable an image to be taken of at least a portion of the capillary component 300 within which a fluid sample has been captured. For example, as illustrated in FIG. 5, an exemplary cartridge housing 100 may include at least one imaging orifice (e.g., a first imaging orifice 114, a second imaging orifice 124) provided at a front surface and/or a rear surface of the cartridge housing 100 such that at least a portion of the capillary component 300 may be visible within a field of view of an imagining device of an exemplary fluid sampling system, as described herein. For example, when the exemplary replaceable fluid sampling cartridge 10 is disposed in an installed position within a fluid sampling system, at least a portion of each imaging orifice 114, 124 of the cartridge housing 100 may be aligned with one or more of a receiving area within which the capillary component 300 is configured to receive a fluid sample, an illumination device of the fluid sampling system, and a field of view (e.g., a line of sight) of an imaging device of the fluid sampling system. For example, an exemplary imaging orifice (e.g., a first imaging orifice 114, a second imagining orifice 124) may embody a window configured such that a light beam emitted from an exemplary illumination source and/or a line of sight defined by an imaging device of a fluid sampling system may extend directly to at least a portion of the capillary component within which a fluid sample has been captured, as described herein, without requiring disassembly of the cartridge housing 100 and/or interruption of the operation of the replaceable fluid sampling cartridge 10.

In various embodiments, the cartridge housing 100 of an exemplary replaceable fluid sampling cartridge 10 may be comprised of a plurality of separable components that fit together to collectively define the cartridge housing 100. For example, as illustrated in FIG. 5, an exemplary cartridge housing 100 may comprise a front housing portion 110 and a rear housing portion 120 that may be detachably fastened relative to one another to collectively define an exterior shell of the cartridge housing 100 and the interior housing portion therein. In various embodiments, the front housing portion 110 may be secured relative to the rear housing portion 120 via any of a variety of fastening configurations, such as, for example, any operable mechanical fastening means, tape (e.g., around a portion of an exterior perimeter of the cartridge housing 100), adhesive, ultrasonic welding, and/or the like.

In such an exemplary configuration, as shown, each of the front and the rear housing portions 110, 120 may define a respective portion of the conduit channel 130, which may embody a partial material recess provided along an interior surface of the respective housing portion that may be configured to receive at least a portion (e.g., half) of an exemplary fluid conduit 200 therein. For example, as illustrated, in various embodiments, the front housing portion 110 of the cartridge housing 100 may define a first conduit channel portion 130a and the rear housing portion 120 of the cartridge housing 100 may define a second conduit channel portion 130b. The first and second conduit channel portions 130a, 130b may have an at least substantially mirrored configuration and may be positioned along the respective housing portions such that, upon coupling of the front housing portion 110 and the rear housing portion 120, the first and second conduit channel portions 130a, 130b are at least substantially aligned with one another to collectively define a conduit channel 130 operable for securing at least a portion of the fluid conduit 200 within the cartridge housing 100 (e.g., in between the front housing portion 110 and the rear housing portion 120).

Further, in various embodiments, the front and the housing portion 110 and the rear housing portion 120 may each define a respective portion of the capillary channel 140, which may embody a partial material recess provided along an interior surface of the respective housing portion that may be configured to receive at least a portion (e.g., half) of an exemplary capillary component 300 therein. For example, as illustrated, in various embodiments, the front housing portion 110 of the cartridge housing 100 may define a first capillary channel portion 140a and the rear housing portion 120 of the cartridge housing 100 may define a second capillary channel portion 140b. The first and second capillary channel portions 140a, 140b may have an at least substantially mirrored configuration and may be positioned along the respective housing portions such that, upon coupling of the front housing portion 110 and the rear housing portion 120, the first and second capillary channel portions 140a, 140b are at least substantially aligned with one another to collectively define a capillary channel 140 operable for securing at least a portion of the capillary component 300 within the cartridge housing 100 (e.g., in between the front housing portion 110 and the rear housing portion 120).

Further, in various embodiments, the front and the housing portion 110 and the rear housing portion 120 may each define a respective imaging orifice configured to facilitate the capturing of an image of a fluid sample disposed within the capillary component 300 secured within the cartridge housing 100. For example, as illustrated in FIG. 5, the front housing portion 110 of an exemplary cartridge housing 100 may include a first imaging orifice 114 extending through a front surface thereof such that at least a portion of the capillary component 300 secured between the front and rear housing portions 110, 120 (e.g., within a capillary channel 140) may be directly accessible to a beam of light emitted from an illumination source of a fluid sampling system without disassembling the cartridge housing 100. As further illustrated, the rear housing portion 120 of the exemplary cartridge housing 100 may include a second imaging orifice 124 extending through a rear surface thereof that is positioned opposite the front surface of the front housing portion 110 such that the at least a portion of the capillary component 300 that is being illuminated by the light beam emitted from the illumination source, as described herein in further detail in reference to FIG. 7, may be visible within a field of view of an imagining device of the exemplary fluid sampling system. For example, when the front and rear housing portions 110, 1120 of the cartridge housing 100 are coupled together and the exemplary replaceable fluid sampling cartridge 10 is provided in an installed position within a fluid sampling system, the first and second imaging orifices 114, 124 may be aligned with each of an illumination device of the fluid sampling system, a field of view (e.g., a line of sight) of an imaging device of the fluid sampling system, and a receiving area defined along the capillary component 300 (e.g., in between the first and second capillary ends 301, 302) within which a fluid sample received by the capillary component 300 may be received, suspended, and/or otherwise positioned during operation of the fluid sampling system.

In various embodiments, as illustrated in FIG. 5, the capillary component 300 of an exemplary replaceable fluid sampling cartridge 10 may be configured to receive at least a portion of the volume of fluid flowing along the fluid conduit 200 (e.g., within an interior conduit portion 200b) to capture a fluid sample from the volume of fluid within a receiving area defined along the capillary component 300. As described herein, the capillary component 300 may be configured to receive, retrieve, capture, and/or otherwise divert a fluid sample from the interior channel portion 200b using a capillary effect and/or a capillary action (e.g., via an at least substantially small opening defined at the first capillary end 301) that causes the fluid sample to be disposed at (e.g., within) a receiving area defined along the capillary component length such that an image of the sample may be captured by an imaging device of an exemplary fluid sampling system. For example, in various embodiments, an exemplary capillary component 300 may comprise an at least partially tubular configuration defined by an interior capillary portion that extends from the first capillary end 301 to the second capillary end 302 and is configured to enable the capture of the fluid sample therein via a capillary effect and/or capillary action. The interior capillary portion may be defined at least in part by a first opening at the first capillary end 301, which may embody a capillary fluid inlet through which the capillary component 300 receives the fluid sample from within the fluid conduit 200. In such an exemplary embodiment, the first opening may be defined by an at least substantially small surface area sufficient to enable the capillary component 300 to capture a fluid sample therethrough using a capillary effect and/or capillary action. Further, an opposite end of the interior capillary portion may be defined at least in part by a second opening at the second capillary end 302, which may embody a capillary outlet through which at least a portion of the air within the interior capillary portion prior to the capturing of the fluid sample may be displaced to enable the capturing of the fluid sample within the capillary component 300.

As illustrated, an exemplary capillary component 300 may define a capillary component length that is defined between a first capillary end 301 and an opposing second capillary end 302. For example, an exemplary replaceable fluid sampling cartridge 10 may be configured such that the first capillary end 301 of the capillary component 300 is in fluid communication with the interior conduit portion 200b of the fluid conduit 200 to enable the conduit component 300 to pull a fluid sample from the volume of fluid flowing through the fluid conduit 200 as it passes the first conduit end 301. [[as illustrated, fluid conduit 200 may define an opening along an intermediate portion thereof (e.g., between the fluid conduit inlet 201 and the fluid conduit outlet 202) through which at least a portion of the capillary component 300, including the first capillary end 301, may be disposed to enable the fluid engagement of the first capillary end 301 with the volume of fluid received by the replaceable fluid sampling cartridge 10 (e.g., via the fluid conduit inlet 201). As described herein, upon being captured, absorbed, and/or otherwise diverted via the captured via the first capillary end 301, the fluid sample may flow from the first capillary end 301 along the capillary component length at least substantially towards the second capillary end 302 via capillary effect and/or a capillary action. The capillary component 300 may be configured such that the flow of the fluid sample received via the first capillary end 301 is stopped along the capillary component length prior to the fluid sample reaching the second capillary end 302. As such, at least a portion of the fluid sample captured by the capillary component remains suspended at an intermediate portion of the capillary component 300 defining a receiving area 303. In various embodiments, the receiving area 303 may be defined by the intermediate portion of the capillary component 300 that, upon the capillary component 300 being installed within the cartridge housing 100, is aligned with the first and second imaging orifices 114, 124 of the cartridge housing 100 such that the portion of the fluid sample captured by the capillary component 300 that is suspended therein may be illuminated and imaged by various components of a fluid sampling system, as described herein, to facilitate a determination of one or more fluid characteristics thereof.

For example, the example capillary component 300 may comprise an acetate fiber material. In some embodiments, the acetate fiber material of the example capillary component 300 may diffuse or disperse a fluid sample (e.g., a liquid substance) when the fluid sample is in contact with, injected to, and/or absorbed by the acetate fiber material, and/or may expand the surface area of the fluid sample. In some embodiments, the acetate fiber material of the example capillary component 300 may provide capillary effect or capillary action on the fluid sample.

Additionally, or alternatively, the example capillary component 300 may comprise an at least substantially transparent (e.g., optically clear) material configured to allow a light beam emitted from an illumination source to pass through the walls of the capillary component 300 (e.g., at least substantially towards the imaging device) without causing the light beam to be distorted. For example, in some embodiments, the capillary component 300 may be made of a glass material, a plastic material, and/or any other at least substantially transparent material capable of facilitating operation of the replaceable fluid sampling cartridge as described herein. The example capillary component 300 may diffuse or disperse a fluid sample (e.g., a liquid substance) when the fluid sample is in contact with, injected to, and/or absorbed by the material, and/or may expand the surface area of the fluid sample. In some embodiments, as described herein, the capillary component 300 is made of a material configured to enable a capillary effect or capillary action with respect to the fluid sample.

In various embodiments, the fluid conduit 200 of an exemplary replaceable fluid sampling cartridge 10 may embody an at least substantially tubular component configured for receiving a volume of fluid and defining a fluid flow path through an interior conduit portion 200b defined by the conduit sidewall 200a along which the volume of fluid received by the replaceable fluid sampling cartridge 10 (e.g., at the fluid conduit inlet 201) travels in a flow direction toward the fluid conduit outlet 202 of the fluid conduit 200. As non-limiting examples, in various embodiments, the fluid conduit 200 may be made of an at least substantially rigid material, such as, for example, a plastic material, a glass material, a metal material, and/or the like, or any combination thereof, and/or an at least substantially non-rigid material, such as, for example, a nylon material, a vinyl material, and/or the like, or any combination thereof. In various embodiments, the fluid conduit 200 may define an interior conduit portion 200b defined by an at least substantially uniform cross-sectional area throughout the conduit length of the fluid conduit 200 (e.g., as defined between the fluid conduit inlet 201 and the fluid conduit outlet 202). As an illustrative example, in various embodiments, the cross-sectional area of the interior conduit portion 200b of the fluid conduit 200 may be at least approximately between 0.1 mm² and 3.0 mm² (e.g., between 0.15 mm² and 0.5 mm²).

As illustrated in FIG. 5, in various embodiments, a fluid conduit 200 may define a capillary opening 203 extending through the conduit sidewall 200a at an intermediate location along the conduit length of the fluid conduit 200 defined in between the fluid conduit inlet 20 and the fluid conduit outlet 202. For example, the capillary opening 203 may extend through the conduit sidewall 200a in an at least substantially perpendicular direction relative to the central axis of the fluid conduit 200. In various embodiments, the capillary opening 203 may have a configuration (e.g., a shape) that corresponds to that of a cross-section of the capillary component 300 such that the capillary opening 203 is configured for receiving at least a portion of the capillary component 300 therethrough. In various embodiments, the fluid conduit 200 may be configured for facilitating a fluid connection between the interior conduit portion 200b and the first capillary end 301 of the capillary component 300.

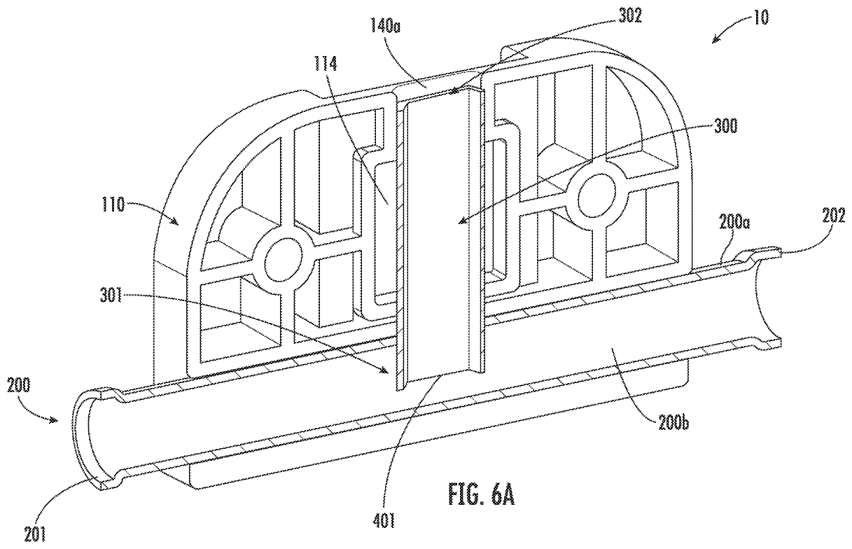

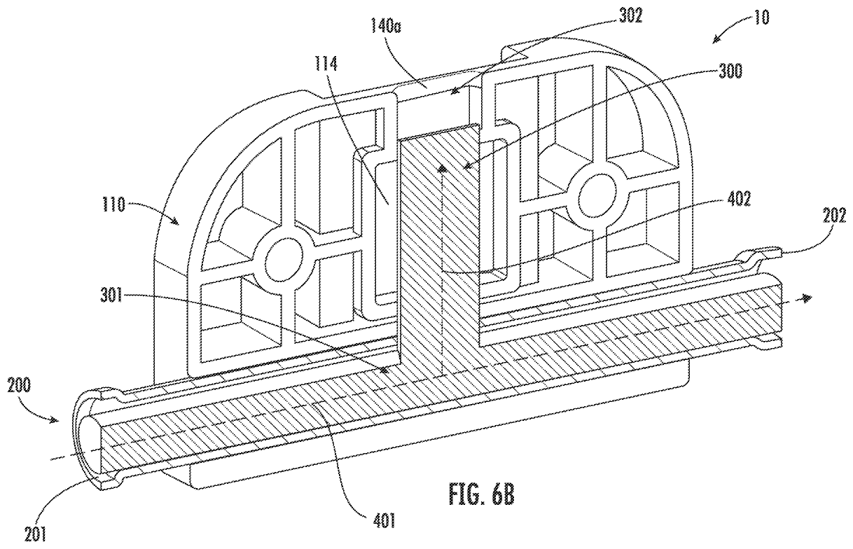

As shown in the exemplary replaceable fluid sampling cartridge 10 illustrated in FIGS. 6A and 6B, in various embodiments, the fluid conduit 200 may be configured to receive the capillary component 300 within the capillary opening 203 such that a first portion of the capillary component 300 including the first capillary end 301 (e.g., a first capillary component length) is disposed within the interior conduit portion 200b, while a second portion of the capillary component 300 including the second capillary end 302 (e.g., a second capillary component length) is provided at an external position relative to the conduit sidewall 200a. FIG. 6A and FIG. 6B illustrate cross-sectional views of an example replaceable fluid sampling cartridge according to various exemplary embodiments described herein. In particular, FIG. 6A provides a cross-sectional view of an exemplary replaceable fluid sampling cartridge 10 in a non-operational condition according to various embodiments described herein. Further, FIG. 6B provides a cross-sectional view of an exemplary replaceable fluid sampling cartridge 10 in an operational condition according to various embodiments described herein. In such an exemplary configuration, the capillary opening 203 defined by the fluid conduit 200 is configured to enable an arrangement of the first capillary end 301 of the capillary component 300 along the fluid flow path defined by the fluid conduit 200 while the second capillary end 302 is positioned outside of the fluid conduit 200 such that that a receiving area defined between the first and second capillary ends 301, 302 is accessible, visible, and/or otherwise at least partially exposed for purposes of capturing one or more images thereof (e.g., via an imaging device of the fluid sampling system).

As illustrated, in various embodiments, an exemplary replaceable fluid sampling cartridge 10 may be configured such that, upon the capillary component 300 being provided within the capillary opening 203 and secured in position by the cartridge housing 100, the capillary component 300 may define an at least substantially perpendicular arrangement relative to the fluid conduit 200. In such an exemplary embodiment, the capillary component length defined by the capillary component 300 may extend from the first capillary end 301 disposed at an intermediate location along the conduit length of the fluid conduit 200 perpendicularly away from the conduit sidewall 200a of the fluid conduit 200 to the second capillary end 302 positioned at least substantially adjacent a third opening defined along a top surface of the cartridge housing 100.

Referring now to FIG. 6B, the exemplary replaceable fluid sampling cartridge 100 is configured for installation and operation within a fluid sampling system such that the replaceable fluid sampling cartridge 10 receives a volume of fluid from an upstream portion of the fluid sampling system at a fluid conduit inlet 201 defined by the fluid conduit 200. For example, the cartridge housing 100 (e.g., the first housing portion 110, as illustrated) may be configured to secure the fluid conduit 200 in an arrangement wherein the fluid conduit inlet 201 is disposed at least substantially adjacent a first lateral side of the cartridge housing 100. The fluid conduit 200 may be configured to direct the volume of fluid along a fluid flow path defined within the interior conduit portion of the fluid conduit 200 in a first fluid flow direction 401 toward the fluid conduit outlet 202. As illustrated, the fluid conduit 200 may be arranged relative to the capillary component 300 such that the volume of fluid received by the fluid conduit 200 may pass the first capillary end 301 fluidly connected with the interior conduit portion 200b as it flows from the fluid conduit inlet 201 in the first fluid flow direction 401. In various embodiments, at least a portion of the volume of fluid may be redirected from the fluid conduit 200 into the capillary component 300 in a second fluid flow direction 402 as the volume of fluid flows past and/or at least substantially adjacent to the first capillary end 301 (e.g., a first opening defined at the first capillary end 301). For example, in various embodiments, the fluid conduit 200 may be arranged relative to the capillary component 300 such that the first fluid flow direction 401 is at least substantially perpendicular to capillary component length of the capillary component 300 and/or the second fluid flow direction 402.

For example, as illustrated in FIG. 6B, the capillary component 300 may be configured such that as the volume of fluid flows in the first fluid direction 401 past the first capillary end 301, at least a portion of the volume of fluid is pulled towards the second fluid direction 402 as a result of a capillary effect and/or capillary action defined by the capillary component 300. In various embodiments, the portion of the volume of fluid that is redirected (e.g., via the capillary effect) towards the second fluid flow direction 402 through the first capillary end 301 and captured within the capillary component 300 may define the fluid sample that is captured by the replaceable fluid sampling cartridge 10 during operation of the fluid sampling system. As described herein, the exemplary replaceable fluid sampling cartridge 10 may be configured such that the captured fluid sample may be imaged from its position within the capillary component 300 (e.g., within the capillary component at a location corresponding to the receiving area positioned within the field of view of the imaging device) by an imaging device of the fluid sampling system. [[For example, the capillary component 300 may be configured to capture, via a capillary effect and/or capillary activity, a fluid sample defined by a volume that sufficiently large such that at least a portion of the fluid sample continues to travel within the capillary component 300 in the second fluid flow direction 402 until at least a portion of the fluid sample is disposed within the receiving area defined by the capillary component 300. Further, in various embodiments, the exemplary replaceable fluid sampling cartridge 10 may be configured such that the portion of the volume of fluid flowing through the fluid conduit 200 that is not redirected into and/or captured by the capillary component 300 may continue to flow along the fluid flow path defined within the interior conduit portion in the first fluid flow direction 401 to the fluid conduit outlet 202, where it may be dispensed from the replaceable fluid sampling cartridge 10 to a downstream portion of the fluid sampling system fluidly connected therewith, such as, for example, a drain line.

FIG. 7 illustrates an isolated perspective view of various components of a fluid sampling system and an example replaceable fluid sampling cartridge in accordance with some example embodiments described herein. In particular, FIG. 7 illustrates an exemplary replaceable fluid sampling cartridge 100 positioned in an installed position relative to an imaging device and an illumination device of an exemplary fluid sampling system. For example, as illustrated in FIG. 7, a fluid sampling system configured for utilizing an exemplary replaceable fluid sampling cartridge 10 to capture a fluid sample from a volume of fluid may comprise an illumination device 72 and an imaging device 73. For example, the exemplary replaceable fluid sampling cartridge 10 may be provided in an installed position within the fluid sampling system (e.g., relative to the illumination device 27 and/or the imagining device 73) to facilitate execution of a fluid sample collection operation, wherein a fluid sample is captured within a capillary component 300 of the replaceable fluid sampling cartridge 10. Further, the fluid sampling system may be configured to utilize the illumination device 72 and the imaging device 73 to facilitate execution of a fluid sample analysis operation, wherein an image of at least a portion of the fluid sample captured within the capillary component 300 of the replaceable fluid sampling cartridge 10 is captured by the imaging device 73 and further analyzed by the fluid sampling system (e.g., a controller) to determine at least one fluid characteristic of the fluid sample, such as, for example, white blood cell type, white blood cell concentration, and/or the like.

In various embodiments, an exemplary fluid sampling system may comprise at least one illumination device. In the exemplary embodiment shown in FIG. 7, an exemplary illumination device 72 is illustrated. For example, the illumination device 72 may be configured to produce, generate, emit, and/or trigger the production, generation, and/or emission of light. The example illumination device 72 may include, but is not limited to, laser diodes (for example, UV, visible, or IR laser diodes, edge-emitting laser diodes, surface-emitting laser diodes, and/or the like). Additionally, or alternatively, the illumination device 72 may comprise one or more light-emitting diodes (LEDs). Additionally, or alternatively, the illumination device 72 may comprise one or more other forms of natural and/or artificial sources of light.

In some embodiments, at least one illumination device is configured to emit at least one light beam. In some embodiments, the at least one light beam emitted by the at least one illumination device may comprise coherent light. In the present disclosure, the term “coherent light” refers to a light beam where the wavefront has a synchronized phase. Examples of coherent light include, but are not limited to, laser light. For example, the light beam in laser light has the same frequency and phase. In some embodiments, to emit coherent light, the at least one illumination device includes, but is not limited to, laser diodes (for example, UV, visible, or IR laser diodes, edge-emitting laser diodes, surface-emitting laser diodes, and/or the like).

In some embodiments, the at least one light beam emitted by the at least one illumination device may comprise incoherent light or at least partially incoherent light. In the present disclosure, the term “incoherent light” (or “low coherence light” as used interchangeably herein) refers to a light beam where the wavefront does not have a synchronized phase. For example, incoherent light does not contain photons with the same frequency and does not have wavelengths that are in phase with one another. In some embodiments, to emit incoherent light, the at least one illumination device includes, but is not limited to, light-emitting diodes (LEDs).

In various embodiments, an exemplary fluid sampling system may be configured such that at least one light beam emitted by the illumination device 72 is directed towards the capillary component 300 of a replaceable fluid sampling cartridge 100 disposed in an installed position. As described herein, the capillary component 300 may comprise a transparent material that enables the at least one light beam emitted from the illumination device 72 to pass therethrough. For example, the at least one light beam may further pass through the fluid sample captured within the capillary component 300 (e.g., a portion of the fluid sample disposed within the receiving area defined by the capillary component 300).

In some embodiments, the imaging device 73 comprises an image sensor configured to capture at least one image of a fluid sample captured within the capillary component 300 of the replaceable fluid sampling cartridge 10. For example, the imaging device may generate digital holography image data associated with the fluid sample suspended within the receiving area of the capillary component 300.

In the present disclosure, the term “digital holography image data” refers to image data that is generated based on digital holography techniques, including, but not limited to, lensless holography techniques. For example, the digital holography image data may be generated by the image sensor without any imaging lenses and without any adjustments. In such an example, there are no imaging lenses between the bottom surface of the flow chamber device 404 and the image sensor. The digital holography image data may comprise a digital holography image of the fluid sample (for example, a digital holography image of various particles, cells, etc. in the fluid sample). In some embodiments, the digital holography image is blurry and/or out of focus, and example embodiments of the present disclosure may generate focused images associated with the fluid sample based at least in part on the digital holography image, details of which are described herein.

In some examples, the image sensor may comprise one or more imagers and/or image sensors. Various examples of the image sensor may include, but are not limited to, a charge-coupled device (CCD), a complementary metal-oxide semiconductor (CMOS) sensor, and/or the like. As described above, in some embodiments, the image sensor does not comprise any lenses so as to generate digital holography image data based on lensless holography techniques.

While the description above provides an example of implementing digital holography techniques, it is noted that the scope of the present disclosure is not limited to the description above. In some examples, an example fluid sample imaging system may implement other imaging techniques. For example, example embodiments of the present disclosure may implement optical microscopy as the imaging technique. Additionally, or alternatively, example embodiments of the present disclosure may implement ultraviolet (UV) fluorescence as the imaging technique.

While the exemplary embodiment illustrated in FIG. 7 provides an example positional arrangement between the illumination device 72, the imaging device 73, and an exemplary replaceable fluid sampling cartridge 10 in an installed position, it is noted that the scope of the present disclosure is not limited to the description above. In some examples, the illumination device 72, the imaging device 73, and/or the replaceable fluid sampling cartridge 10 may be positioned differently than those shown in FIG. 7.

As described herein, the replaceable fluid sampling cartridge 10 may be positioned in an installed position wherein the replaceable fluid sampling cartridge 10 is at least substantially adjacent (e.g., in contact with or spaced a distance away from) the imaging device 73 of the fluid sampling system such that the imaging device 73 may effectively capture one or more images of the fluid sample captured within the capillary component 300 of the replaceable fluid sampling cartridge 10. As discussed herein, the replaceable fluid sampling cartridge 10 may be replaceable such that the replaceable fluid sampling cartridge 10 may be inserted into and/or removed from the installed position defined relative to the imaging device 73 and/or the illumination device 72 of the fluid sampling system. In various embodiments, a replaceable fluid sampling cartridge 10 may be configured to facilitate a removal thereof from the installed position within the fluid sampling system upon execution of the fluid sample analysis operation. For example, upon the replaceable fluid sampling cartridge 10 having captured a fluid sample within the capillary component 300 and one or more images of the fluid sample having been captured by the imaging device 73, the replaceable fluid sampling cartridge 10 may be removed from the installed position (e.g., removed from within a housing defined by the fluid sampling system) and disposed or otherwise transported to a secondary location. For example, the removed replaceable fluid sampling cartridge 10 may be replaced with a different, unused replaceable fluid sampling cartridge 10, which may be utilized in executing a second (e.g., subsequent) fluid sample collection operation.

In various embodiments, an exemplary fluid sampling system may include a housing that defines one or more alignment features, support features, and/or the like configured to engage at least a portion of the cartridge housing 100 of the replaceable fluid sampling cartridge 10 to maintain a desired positioning of the replaceable fluid sampling cartridge 10 in the installed position relative to the imaging device 73. Such alignment features and/or support features may comprise one or more grooves, slots, ridges, and/or the like configured to position the replaceable fluid sampling cartridge 10 in a desired position relative to the imaging device 73. For example, the installed position defined by the replaceable fluid sampling cartridge 10, as described herein, may be defined by one or more surfaces of the cartridge housing 100 being in physical contact with a portion of the alignment features defined by the fluid sampling system such that the first and second imaging orifices defined by the cartridge housing 100 and the portion of the capillary component 300 aligned therewith (e.g., the receiving area) are at least substantially aligned with both the imaging device 73 and the illumination device 72. As illustrated, in various embodiments, the replaceable fluid sampling cartridge 10 may be configured for arrangement in an installed position within the fluid sampling system such that a light beam 72a emitted from the illumination device 72 may travel towards the cartridge housing 100 and be received through an imagining orifice defined by the rear housing portion 120 of the cartridge housing 100. In such an exemplary circumstance, the replaceable fluid sampling cartridge 10 may be configured such that the light beam 72a passing through the imaging orifice defined by the rear housing portion 120 may directly engage a rear surface of the capillary component 300 at a receiving area defined thereby, such that a fluid sample captured within the capillary component 300 (e.g., at the receiving area) is at least partially illuminated by the light beam 72a. Further, the replaceable fluid sampling cartridge 10 may be configured for arrangement in the installed position such that the imaging device 73 has a direct line of sight through an imaging orifice defined by the front housing portion 110 to a front surface of the capillary component 300 (e.g., at the receiving area). For example, the replaceable fluid sampling cartridge 10 may be configured for positioning in an installed position defined at a predetermined distance away from the imaging device 73 that is calibrated to optimize the fluid analysis functions of fluid sampling system with respect to the fluid sample captured by the replaceable fluid sampling cartridge 10, as described herein.

Many modifications and other embodiments of the present disclosure set forth herein will come to mind to one skilled in the art to which these embodiments pertain having the benefit of the teachings presented in the foregoing descriptions and the associated drawings. Therefore, it is to be understood that the disclosure are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1. A fluid sampling cartridge for capturing a fluid sample, the fluid sampling cartridge comprising:
a fluid conduit having an interior conduit portion defined between a fluid conduit inlet and a fluid conduit outlet of the fluid conduit, wherein the fluid conduit is configured to receive a volume of fluid at the fluid conduit inlet such that the fluid conduit defines a fluid flow path within the interior conduit portion between the fluid conduit inlet and the fluid conduit outlet;
a capillary component having a first capillary end in fluid communication with the interior conduit portion and configured to receive at least a portion of the volume of fluid, the at least a portion of the volume of fluid received by the capillary component from the fluid conduit defining a fluid sample; and
a cartridge housing defining an interior housing portion, wherein the cartridge housing is configured for engagement with the fluid conduit and the capillary component such that at least a portion of the fluid conduit and at least a portion of the capillary component are disposed within the interior housing portion.

2. The fluid sampling cartridge of claim 1, wherein the cartridge housing is configured for selective arrangement in an installed position relative to a fluid sampling system, the installed position being defined by an arrangement of the capillary component relative to an imaging device of the fluid sampling system that enables the imaging device to capture imaging data associated with the fluid sample received within the capillary component.

3. The fluid sampling cartridge of claim 1, wherein the cartridge housing comprises a conduit channel defined within the interior housing portion, the conduit channel being configured to receive at least a portion of the fluid conduit therein to secure the fluid conduit relative to the cartridge housing.

4. The fluid sampling cartridge of claim 1, wherein the cartridge housing comprises a capillary channel defined within the interior housing portion, the conduit channel being configured to receive at least a portion of the capillary component therein to secure the capillary component relative to the cartridge housing.

5. The fluid sampling cartridge of claim 4, wherein the capillary channel is positioned such that at least a portion of the capillary component provided therein is at least substantially adjacent to a capillary opening defined by the fluid conduit to facilitate the fluid communication between the capillary component and the fluid conduit.

6. The fluid sampling cartridge of claim 1, wherein the cartridge housing defines one or more imaging orifice configured such that at least a portion of the capillary component is visible through the cartridge housing via the one or more imaging orifice.

7. The fluid sampling cartridge of claim 6, wherein the one or more imaging orifice intersects at least a portion of a capillary channel defined by the cartridge housing, the conduit channel being configured to receive at least a portion of the capillary component therein to secure the capillary component relative to the cartridge housing.

8. The fluid sampling cartridge of claim 6, wherein the one or more imaging orifice includes a first imaging orifice provided on a front surface of the conduit housing and a second imaging orifice provided on a rear surface of the conduit housing.

9. The fluid sampling cartridge of claim 8, wherein the first imaging orifice is positioned at least substantially adjacent a front capillary surface of the capillary component disposed within the interior housing portion of the cartridge housing such that the front capillary surface is visible through the first imaging orifice, and wherein the second imaging orifice is positioned at least substantially adjacent a rear capillary surface of the capillary component such that the rear capillary surface is visible through the second imaging orifice.

10. The fluid sampling cartridge of claim 1, wherein the cartridge housing is defined by a plurality of detachable components configured for selective attachment relative to one another to collectively define the cartridge housing.

11. The fluid sampling cartridge of claim 10, wherein the cartridge housing comprises a front housing portion and a rear housing portion that are configured to be detachably fastened relative to one another to collectively define an exterior shell of the cartridge housing with the interior housing portion defined therein.

12. The fluid sampling cartridge of claim 11, wherein the front housing portion defines a first conduit channel portion and the rear housing portion defines a second conduit channel portion, the first conduit channel portion and the second conduit channel portion collectively defining a conduit channel within the interior housing portion that is configured for receiving at least a portion of the fluid conduit.

13. The fluid sampling cartridge of claim 11, wherein the front housing portion defines a first capillary channel portion and the rear housing portion defines a second capillary channel portion, the first capillary channel portion and the second capillary channel portion collectively defining a capillary channel within the interior housing portion that is configured for receiving at least a portion of the capillary component.

14. The fluid sampling cartridge of claim 11, wherein the front housing portion defines a first imaging orifice and the rear housing portion defines a second imaging orifice, and wherein at least a portion of the capillary component defining a receiving area within which the capillary component captures the fluid sample is positioned in between the first imaging orifice and the second imaging orifice.

15. The fluid sampling cartridge of claim 1, wherein the fluid conduit defines a capillary opening extending through the conduit sidewall at an intermediate location defined between the fluid conduit inlet and the fluid conduit outlet of the fluid conduit, the capillary opening being configured for receiving at least a portion of the capillary component therethrough.

16. The fluid sampling cartridge of claim 1, wherein the capillary component comprises a first opening defined at a first capillary end and a second opening defined at a second capillary end, wherein the first opening defines a fluid inlet through which the capillary component receives the fluid sample.

17. The fluid sampling cartridge of claim 16, wherein the capillary component is arranged relative to the fluid conduit such that the first opening of the capillary component is disposed within the interior conduit portion of the fluid conduit.

18. The fluid sampling cartridge of claim 1, wherein the fluid sampling cartridge is configured for arrangement within fluid sampling system such that the fluid sample captured within the capillary component is disposed within a field of view of an imaging device.

19. The fluid sampling cartridge of claim 18, wherein the fluid sampling cartridge is configured to enable an imaging of the fluid sample captured within the capillary component by the imaging device at an instance during operation of the fluid sampling system in which the volume of fluid is flowing through the fluid conduit.

20. The fluid sampling cartridge of claim 1, wherein the capillary component defines an at least substantially perpendicular arrangement relative to the fluid conduit.
